(19) United States
(12) Patent Application Publication
(10) Pub. No.: US 2024/0173164 A1
(43) Pub. Date: May 30, 2024

(54) Therapy Device with Cold-Heat Exchange Structure

(71) Applicant: ODECO LTD., Shenzhen (CN)

(72) Inventor: Huiming ZHONG, Shenzhen (CN)

(21) Appl. No.: 18/525,192

(22) Filed: Nov. 30, 2023

(30) Foreign Application Priority Data
Nov. 30, 2022 (CN) 202223218935.6

(51) Int. Cl. A61F 7/00

(52) U.S. Cl. CPC A61F 7/007; A61F 2007/0075; A61F 2007/0078

(57) ABSTRACT

A therapy device with a cold-heat exchange structure comprises a housing, a cold-heat stimulation area for performing cold-heat stimulation therapy on body parts, and a cooling and heating assembly physically connected to the cold-heat stimulation area. The cooling and heating assembly comprises at least one semiconductor thermoelectric cooling element and at least one heat dissipation element, the semiconductor thermoelectric cooling element has a first end and a second end, the first end is connected to the heat dissipation element, and the second end is physically connected to the cold-heat stimulation area. The therapy device can perform cold stimulation therapy, heat stimulation therapy or cold and heat stimulation alternate therapy on body parts by means of the cold-heat stimulation area during vibratory massage, thus greatly improving the comfort in use and increasing blood circulation of massaged/therapeutic parts.

### BACKGROUND OF THE PRESENT INVENTION

The invention relates to a massage therapy device with a cold-heat exchange structure.

### DESCRIPTION OF RELATED ARTS

In the field of adjuvant rehabilitation treatment, therapy devices, as equipment for performing massage therapy on body parts of patients, can realize massage therapy of many parts of patients to provide adjuvant rehabilitation treatment for patients who suffer from hand and leg numbness and have difficulty in walking.

Existing therapy devices have only one function, can merely mechanically massage body parts of patients, and cannot realize cold-heat stimulation therapy, thus being unsatisfying in massage therapy effect and narrow in application range.

### SUMMARY OF THE PRESENT INVENTION

The objective of the invention is to provide a therapy device with a cold-heat exchange structure, which can realize cold-heat stimulation therapy.

The objective of the invention is realized by designing a therapy device with a cold-heat exchange structure, comprising: a housing, a cold-heat stimulation area for performing cold-heat stimulation therapy on body parts, and a cooling and heating assembly physically connected to the cold-heat stimulation area, wherein:
the cooling and heating assembly comprises at least one semiconductor thermoelectric cooling element and at least one heat dissipation element, the semiconductor thermoelectric cooling element has a first end and a second end for producing heat transfer when the semiconductor thermoelectric cooling element is powered on, the first end is connected to the heat dissipation element, and the second end of the semiconductor thermoelectric cooling element is physically connected to the cold-heat stimulation area to transfer a cold-heat transfer effect to the cold-heat stimulation area.

Further, the cooling and heating assembly is arranged in the housing.

Further, at least part of the heat dissipation element is exposed onto the housing.

Further, part of the housing forms the heat dissipation element.

Further, the therapy device with a cold-heat exchange structure further comprises a soft colloid, wherein the soft colloid is provided with a massage cavity and covered with at least part of the housing, an opening is formed in an end of the massage cavity, and the cold-heat stimulation area is arranged on a cavity wall of the massage cavity.

Further, an end face of the second end of the semiconductor thermoelectrical cooling element faces the massage cavity.

Further, the heat dissipation element is made of a metal material or a thermally conductive plastic, and a size of the semiconductor thermoelectrical cooling element is not greater than that of the heat dissipation element.

Further, the semiconductor thermoelectrical cooling element is configured as a sheet structure, the first end and the second end of the semiconductor thermoelectrical cooling element have end faces, and the end face of the first end is attached and connected to the heat dissipation element.

Further, the therapy device with a cold-heat exchange structure further comprises a heat conduction element attached and connected to an end face of the second end of the semiconductor thermoelectrical cooling element, and the semiconductor thermoelectrical cooling element and the cold-heat stimulation area are connected through the heat conduction element.

Further, at least part of the heat conduction element forms the heat-cold stimulation area.

Further, the therapy device with a cold-heat exchange structure further comprises a soft rubber sleeve which is at least partially arranged on an outer surface of the housing.

Further, the therapy device with a cold-heat exchange structure further comprises a vibration motor, a battery and a PCBA, wherein the vibration motor is arranged in the housing, the battery is electrically connected to the PCBA, and the PCBA is electrically connected to the cooling and heating assembly and/or the vibration motor.

The therapy device can perform cold stimulation therapy, heat stimulation therapy or cold and heat stimulation alternate therapy on body parts by means of the cold-heat stimulation area during vibratory massage, thus greatly improving the comfort in use and increasing the blood circulation of massaged/therapeutic parts. The therapy device integrates the vibratory massage function and the cold-heat stimulation therapy function, is convenient to use and wide in application range, and has a variety of functions

### BRIEF DESCRIPTION OF THE DRAWINGS

FIG. 1 is a sectional view of a first preferred embodiment of the invention;

FIG. 2 is an exploded view of the first preferred embodiment of the invention;

FIG. 3 is a schematic diagram of a semiconductor thermoelectric cooling element according to the first preferred embodiment of the invention;

FIG. 4 is an exploded view of a second preferred embodiment of the invention;

FIG. 5 is a sectional view of a structural A of the second preferred embodiment of the invention;

FIG. 6 is a sectional view of a structural B of the second preferred embodiment of the invention.

Reference signs: 1, housing; 2, cold-heat stimulation area; 3, cooling and heating assembly; 31, semiconductor thermoelectric cooling element; 311, first end; 312, second end; 32, heat dissipation element; 4, soft rubber sleeve; 5, vibration motor; 6, battery; 7, PCBA; 8, soft colloid; 81, massage cavity; 82, opening.

### DETAILED DESCRIPTION OF THE PREFERRED EMBODIMENT

The invention will be further described below in conjunction with embodiments.

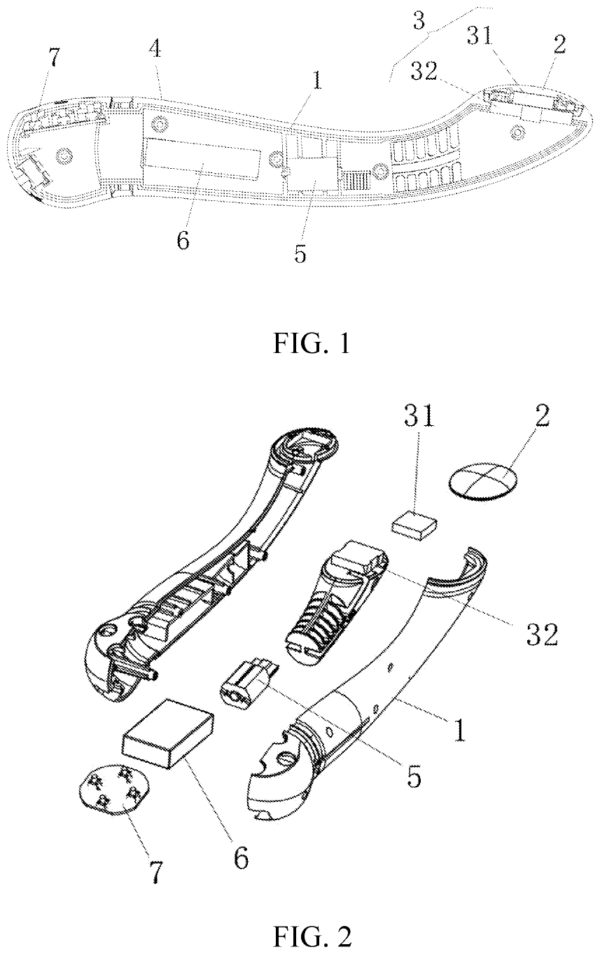

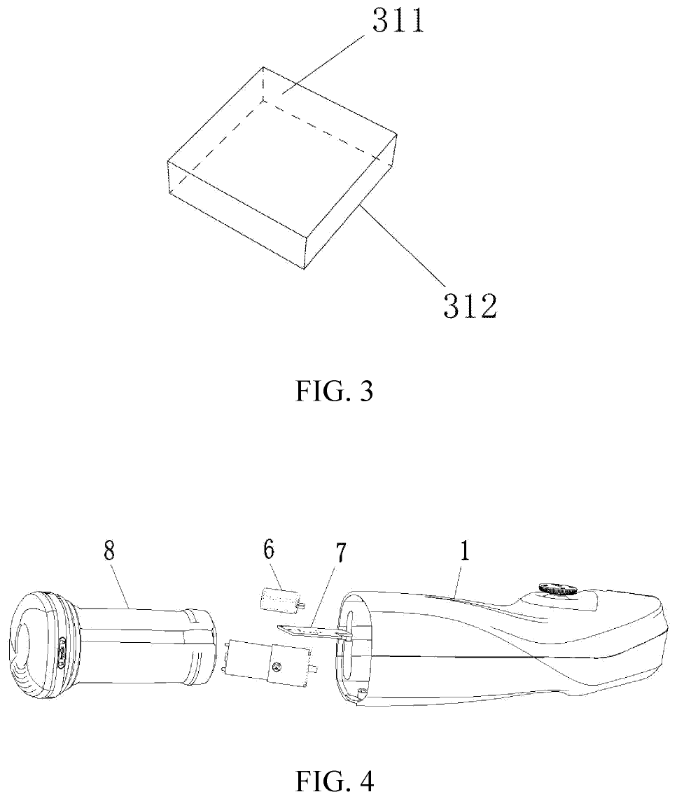

As shown in FIG. 1 and FIG. 2, a therapy device comprises a housing 1, a cold-heat stimulation area 2 and a cooling and heating assembly 3.

The cold-heat stimulation area 2 is arranged on an outer surface of the housing 1 and is configured to perform cold-heat stimulation therapy on body parts; the cooling and heating assembly 3 is arranged in the housing 1 and at least partially located on the outer surface of the housing 1, the cooling and heating assembly 3 comprises at least one semiconductor thermoelectric cooling element 31 and at least one heat dissipation element 32, the semiconductor thermoelectric cooling element 31 has a first end 311 and a second end 312 which, when the semiconductor thermoelectric cooling element 31 is powered on, produce heat transfer and correspond to each other, the first end 311 is connected to the heat dissipation element 32, and the second end 312 of the semiconductor thermoelectric cooling element transfers a heat-cold transfer effect to the cold-heat stimulation area 2. The therapy device further comprises a vibration motor 5 arranged in the housing 1. The therapy device further comprises a battery 6 and a PCBA 7, wherein the battery 6 and the PCBA 7 are both arranged in the housing 1, the battery 6 is electrically connected to the PCBA 7, and the PCBA 7 is electrically connected to the cooling and heating assembly 3 and/or the vibration motor 5. The battery 6 provides driving power for the therapy device.

At least the second end 312 of the semiconductor thermoelectric cooling element is located on the outer surface of the housing 1. The heat dissipation element 32 is made from a metal material or a thermally conductive plastic, wherein the metal material includes, but is not limited to, an aluminum heat dissipation piece. The size of the semiconductor thermoelectric cooling element 31 is not greater than that of the heat dissipation element 32.

In one embodiment, the therapy device further comprises a soft rubber sleeve 4 which is at least partially arranged on the housing. The soft rubber sleeve 4 is preferably a silica gel sleeve, such that the therapy device has a sense of touch similar to human skin and is waterproof.

In one embodiment, the semiconductor thermoelectric cooling element 31 is configured as a sheet structure, the first end 311 and the second end 312 of the semiconductor thermoelectric cooling element have end faces, and the end face of the first end 311 is attached and connected to the heat dissipation element 32. By means of the Peltier effect of semiconductor materials, when a direct current passes through an electric coupling formed by different semiconductor materials connected in series, heat transfer happens between two ends of the electric coupling, that is, one end of the semiconductor thermoelectric cooling element is cold, and the other end of the semiconductor thermoelectric cooling element is hot, such that the cooling purpose is realized by the semiconductor thermoelectric cooling element 31.

In one embodiment, the therapy device further comprises a heat conduction element, wherein the heat conduction element is attached and connected to the end face of the second end of the semiconductor thermoelectric cooling element, and the semiconductor thermoelectric cooling element and the cold-heat stimulation area are connected through the heat conduction element.

In one embodiment, at least part of the heat conduction element forms the cold-heat stimulation area.

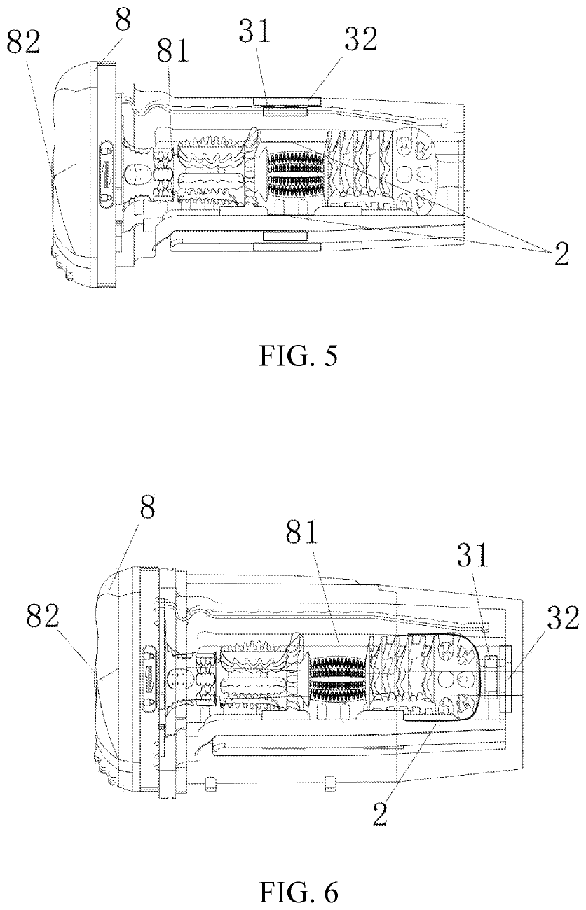

As shown in FIG. 4 and FIG. 5, in another embodiment, the therapy device further comprises a soft colloid 8, wherein the soft colloid 8 is covered with at least part of the housing 1 and is provided with a massage cavity 81, an opening 82 is formed in one end of the massage cavity 81, and the cold-heat stimulation area 2 is arranged in a cavity wall of the massage cavity 81. The heat dissipation element 32 is arranged on the outer surface of the housing 1 to facilitate heat dissipation. If the housing 1 placed in a shell, a heat dissipation and conduction element may be attached to the shell, and the heat dissipation element 32 transfers heat or cold out of the shell by means of the dissipation and conduction element, such that the shell forms a good heat sink.

The heat-cold stimulation area 2 is configured to perform heat-cold massage on body parts. Wherein, the cooling and heating assembly 3 comprises two semiconductor thermoelectric cooling elements 31 which are symmetrically arranged on the soft colloid 8 respectively; the heat-cold stimulation area 2 is located between the two semiconductor thermoelectric cooling elements 31, the heat-cold stimulation area 2 and the semiconductor thermoelectric cooling elements 31 are closely attached to the cavity wall of the massage cavity 81, and the vibration motor 5 is arranged in the housing 1, that is, the vibration motor 5 may be located in the soft colloid 8 and close to the massage cavity 81 or may be located on an outer wall of the soft colloid 8. In this embodiment, the cold-heat stimulation area 2 is located in the massage cavity 81, the soft colloid 8 and the massage cavity 81 thereof are cooled or heated by means of the cooling and heating assembly 3 to perform cold or heat stimulation and vibration therapy on body parts.

As shown in FIG. 6, the therapy device comprises a housing 1, a cold-heat stimulation area 2 and a cooling and heating assembly 3, wherein the cold-heat stimulation area 2 is arranged in the housing 1 and configured to perform cold-heat stimulation therapy on body parts. The therapy device further comprises a soft colloid 8 arranged in the housing 1, the soft colloid 8 is provided with a massage cavity 81, and the cold-heat stimulation area 2 is arranged on a cavity wall of the massage cavity 81; the cooling and heating assembly 3 is arranged in the soft colloid 8; wherein, a first end 311 and a second end 312 of a semiconductor thermoelectric cooling element 31 have end faces, and the end face of the first end 311 is attached and connected to a heat dissipation element 32; at least part of the semiconductor thermoelectric cooling element 31 is inlaid in the soft colloid 8, and the second end 312 of the semiconductor thermoelectric cooling element 31 faces the massage cavity 81; and the second end 312 of the semiconductor thermoelectrical cooling element 31 faces the massage cavity 81, and the end face of the second end 312 is attached to the cavity wall of the massage cavity 81, such that the semiconductor thermoelectric cooling element 31 has a better cold-heat exchange effect.

Heat stimulation and vibration therapy: the semiconductor thermoelectric cooling element 31 is started to heat the cold-heat stimulation area 2, at this moment, the temperature of the surface of the cold-heat stimulation area 2 rises gradually, and the vibration motor 5 works to enable the cold-heat stimulation area 2 to vibrate continuously, thus realizing heat stimulation and vibration therapy of body parts.

Cold stimulation and vibration therapy: the semiconductor thermoelectric cooling element 31 is started to cool the cold-heat stimulation area 2, heat on the surface of the cold-heat stimulation area 2 is transferred out continuously under the action of the heat dissipation element 32 to realize rapid cooling of the cold-heat stimulation area 2, and the vibration motor 5 works to enable the cold-heat stimulation area 2 to vibrate continuously, thus realizing cold stimulation and vibration therapy of body parts. In this way, the blood circulation of massaged/therapeutic parts is increased, and the purposes of relaxing human bodies and relieving fatigue and the feeling of numbness are fulfilled.

The therapy device with a cold-heat exchange structure integrates the vibratory massage function and the cold-heat stimulation therapy function, is convenient to use and wide in application range, and has a variety of functions; mechanical vibrations can be generated by means of the vibration motor to drive the massage part or the cold-heat stimulation area to perform vibratory massage on different parts of human bodies; by adding the cold-heat stimulation therapy function, the therapy device can perform cold or heat stimulation therapy on body parts by means of the cold-heat stimulation area during vibratory massage, thus greatly improving the comfort in use, increasing the blood circulation of massaged/therapeutic parts and fully relaxing human bodies; and the therapy device has a great adjuvant treatment effect for the rehabilitation of patients and is suitable for rehabilitation massage treatment/therapy of body parts such as the legs, the feet, the hands, the waist and the back.

What is claimed is:

1. A therapy device with a cold-heat exchange structure, comprising a housing, a cold-heat stimulation area for performing cold-heat stimulation therapy on body parts, and a cooling and heating assembly physically connected to the cold-heat stimulation area, wherein:
the cooling and heating assembly comprises at least one semiconductor thermoelectric cooling element and at least one heat dissipation element, the semiconductor thermoelectric cooling element has a first end and a second end for producing heat transfer when the semiconductor thermoelectric cooling element is powered on, the first end of the semiconductor thermoelectric cooling element is connected to the heat dissipation element, and the second end of the semiconductor thermoelectric cooling element is physically connected to the cold-heat stimulation area to transfer a cold-heat transfer effect to the cold-heat stimulation area.

2. The therapy device with a cold-heat exchange structure according to claim 1, wherein the cooling and heating assembly is arranged in the housing.

3. The therapy device with a cold-heat exchange structure according to claim 1, wherein at least part of the heat dissipation element is exposed onto the housing.

4. The therapy device with a cold-heat exchange structure according to claim 3, wherein part of the housing forms the heat dissipation element.

5. The therapy device with a cold-heat exchange structure according to claim 1, further comprising a soft colloid, wherein the soft colloid is provided with a massage cavity and covered with at least part of the housing, an opening is formed in an end of the massage cavity, and the cold-heat stimulation area is arranged on a cavity wall of the massage cavity.

6. The therapy device with a cold-heat exchange structure according to claim 5, wherein an end face of the second end of the semiconductor thermoelectrical cooling element faces the massage cavity.

7. The therapy device with a cold-heat exchange structure according to claim 1, wherein the heat dissipation element is made of a metal material or a thermally conductive plastic, and a size of the semiconductor thermoelectrical cooling element is not greater than that of the heat dissipation element.

8. The therapy device with a cold-heat exchange structure according to claim 1, wherein the semiconductor thermoelectrical cooling element is configured as a sheet structure, the first end and the second end of the semiconductor thermoelectrical cooling element have end faces, and the end face of the first end is attached and connected to the heat dissipation element.

9. The therapy device with a cold-heat exchange structure according to claim 1, further comprising a heat conduction element attached and connected to an end face of the second end of the semiconductor thermoelectrical cooling element, wherein the semiconductor thermoelectrical cooling element and the cold-heat stimulation area are connected through the heat conduction element.

10. The therapy device with a cold-heat exchange structure according to claim 9, wherein at least part of the heat conduction element forms the heat-cold stimulation area.

11. The therapy device with a cold-heat exchange structure according to claim 1, further comprising a soft rubber sleeve which is at least partially arranged on an outer surface of the housing.

12. The therapy device with a cold-heat exchange structure according to claim 2, further comprising a vibration motor, a battery and a PCBA, wherein the vibration motor is arranged in the housing, the battery is electrically connected to the PCBA, and the PCBA is electrically connected to the cooling and heating assembly and/or the vibration motor.

13. The therapy device with a cold-heat exchange structure according to claim 2, wherein at least part of the heat dissipation element is exposed onto the housing.

14. The therapy device with a cold-heat exchange structure according to claim 3, further comprising a vibration motor, a battery and a PCBA, wherein the vibration motor is arranged in the housing, the battery is electrically connected to the PCBA, and the PCBA is electrically connected to the cooling and heating assembly and/or the vibration motor.

15. The therapy device with a cold-heat exchange structure according to claim 5, further comprising a vibration motor, a battery and a PCBA, wherein the vibration motor is arranged in the housing, the battery is electrically connected to the PCBA, and the PCBA is electrically connected to the cooling and heating assembly and/or the vibration motor.

16. The therapy device with a cold-heat exchange structure according to claim 7, further comprising a vibration motor, a battery and a PCBA, wherein the vibration motor is arranged in the housing, the battery is electrically connected to the PCBA, and the PCBA is electrically connected to the cooling and heating assembly and/or the vibration motor.

17. The therapy device with a cold-heat exchange structure according to claim 8, further comprising a vibration motor, a battery and a PCBA, wherein the vibration motor is arranged in the housing, the battery is electrically connected to the PCBA, and the PCBA is electrically connected to the cooling and heating assembly and/or the vibration motor.

18. The therapy device with a cold-heat exchange structure according to claim 9, further comprising a vibration motor, a battery and a PCBA, wherein the vibration motor is arranged in the housing, the battery is electrically connected to the PCBA, and the PCBA is electrically connected to the cooling and heating assembly and/or the vibration motor.

19. The therapy device with a cold-heat exchange structure according to claim 11, further comprising a vibration motor, a battery and a PCBA, wherein the vibration motor is arranged in the housing, the battery is electrically connected to the PCBA, and the PCBA is electrically connected to the cooling and heating assembly and/or the vibration motor.
